(19) United States
(12) Patent Application Publication
(10) Pub. No.: US 2007/0224149 A1
(43) Pub. Date: Sep. 27, 2007

(54) Foaming composition

(75) Inventors: Akiko Suzuki, Tokyo (JP); Kentaro Yoshida, Yokohama-shi (JP); Masaaki Yamamoto, Inzai-shi (JP)

Correspondence Address: Ohlandt, Greeley, Ruggiero & Perle, L.L.P., One Landmark Square, 10th Floor, Stamford, CT, 06901-2682, US

(73) Assignee: Kabushiki Kaisha Toshiba

(21) Appl. No.: 11/649,631

(22) Filed: Jan. 4, 2007

(30) Foreign Application Priority Data
Mar. 27, 2006 (JP) 2006-085912

(51) Int. Cl. A61K 8/73

(52) U.S. Cl. 424/70.13

(57) ABSTRACT

A foaming composition is provided, which includes water, pulp, alginic ester, and a surfactant. A preferable example of the alginic ester is propylene glycol alginate. The alginic ester may be mixed therein at a mixing amount ranging from 5 parts by weight to 15 parts by weight based on 100 parts by weight of the pulp.

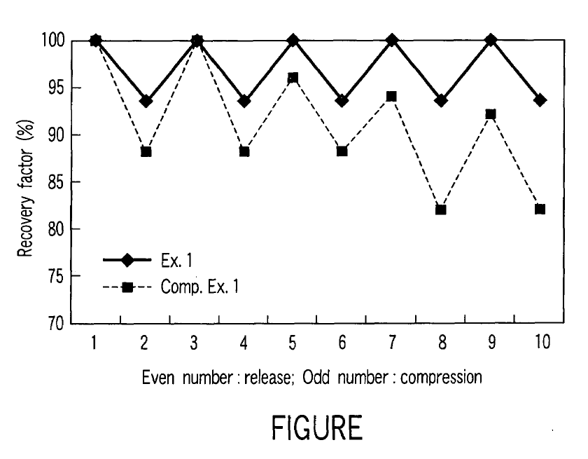

### CROSS-REFERENCE TO RELATED APPLICATIONS

This application is based upon and claims the benefit of priority from prior Japanese Patent Application No. 2006-085912, filed Mar. 27, 2006, the entire contents of which are incorporated herein by reference.

### BACKGROUND OF THE INVENTION

1. Field of the Invention

This invention relates to a foaming composition, a foam and a method of manufacturing a foam.

2. Description of the Related Art

In recent years, in view of protecting the natural environment, it is now demanded to employ biodegradable resins or molded products thereof which can be easily decomposed in the natural environment in place of petroleum-based plastic buffering materials such as foamed polystyrol, etc. Among such biodegradable products, pulp has now been noticed as being useful as a material which is harmless to the environment. Among the conventional pulp foams, those employing a plant-originated binder are considered more preferable as a foam which is harmless to the environment. Alginate which is a plant-originated binder as well as gelatin which is an animal-originated binder is known as being useful as a foaming material. However, since these binders are less effective in stabilizing the cells of foam, it is required to co-use a crosslinking agent. Due to the inclusion of the crosslinking agent however, a foaming material may become difficult to treat or a foam created may become difficult to regenerate.

### BRIEF SUMMARY OF THE INVENTION

A foaming composition according to one aspect of the present invention comprises water; pulp; alginic ester; and a surfactant.

A foam according to another aspect of the present invention comprises pulp; and a binder having cells, the binder binding the pulp and comprising alginic ester and a surfactant.

A method for manufacturing a foam according to a further aspect of the present invention comprises mixing pulp, alginic ester, a surfactant and water to obtain a foaming composition; allowing the foaming composition to bubble, thereby obtaining a foam. precursor; and eliminating water from the foam precursor to form the foam.

### BRIEF DESCRIPTION OF THE SEVERAL VIEWS OF THE DRAWING

The single FIGURE is a graph showing the results of durability test.

### DETAILED DESCRIPTION OF THE INVENTION

Next, embodiments will be explained.

The present inventors have conducted extensive studies on foamed bodies to be produced from the raw materials comprising pulp which is minimal in environmental load or impact and a biomass-originated foaming material such as polysaccharide or protein and found out the following facts. Namely, when alginic ester is employed as a foaming material in the manufacture of a pulp foam, it is possible to maintain a foamed state without crosslinking agent. It is possible, as a result, to realize such a buffering force and such a restoring force that cannot be derived from the conventional biomass-originated foaming materials. Moreover, since the foam thus obtained contains no crosslinking agent, the treatment of foaming material can be facilitated and the regeneration of foam can be easily realized.

The composition according to one embodiment comprises water, pulp, alginic ester, and a surfactant.

As the pulp, it is possible to employ, for example, virgin pulp, regenerated pulp originating from waste paper, etc., bagasse pulp, and straw pulp. These pulps are at first suitably split or refined in advance by a refiner. With respect to the fineness and length of fiber to be obtained from these pulps, there is no particular limitation to them. However, the larger the fineness thereof is, the higher the rigidity of foam to be obtained, and hence the smaller the fineness thereof is, the higher the flexibility of foam to be obtained. Further, the longer the fiber thereof is, the greater the tearing strength of foam becomes, and hence the shorter the fiber length is, the lower the tearing strength of foam becomes.

For example, in the case of softwood pulp, the fiber length thereof should preferably be confined within the range of about 1.5-5.5 mm, and the fineness (diameter) thereof should preferably be confined within the range of about 0.03-0.05 mm.

Alginic ester can be synthesized through the esterification of carboxyl group of alginic acid by alcoholic compounds. Alginic acid is provided with two hydroxyl groups and one carboxyl group. When this carboxyl group is reacted with mono- or polyhydric alcohols or alkylene oxides, alginic ester having ester linkage can be obtained.

As the alcohols, it is possible to employ monohydric alcohols, dihydric alcohols and trihydric alcohols, all comprising an alkyl group having 1 to 5 carbon atoms. In view of hydrophilicity, surface activity and practicability of the product to be obtained, dihydric alcohols are most preferable. Examples of dihydric alcohols include propylene glycol, ethylene glycol, etc.

As the alkylene oxides, it is possible to employ compounds having alkyl group having 1 to 5 carbon atoms. More specifically, it is possible to employ ethylene oxide, propylene oxide, butene oxide, etc. Further, the alkylene oxides may be reacted with a compound having carboxyl group to modify them so as to have an ester linkage. Especially preferable examples of the compound having carboxyl group are an organic acid having 1 to 5 carbon atoms. It is also possible to employ a compound having carboxyl group and hydroxyl group such as lactic acid and glycolic acid.

These alginates may be employed singly or in combination of two or more.

For example, in the case of propylene glycol alginate, a weight average molecular weight thereof should preferably be confined to about 70,000 to 100,000. The degree of polymerization in this case corresponds to about 299 to 427. Generally, as the weight average molecular weight of alginate becomes higher, the viscosity thereof would be proportionally increased, thus making it difficult to dissolve it in water. At the same time, the buffering properties of foam material may more likely be lost. Therefore, the upper limit of the weight average molecular weight of the alginic ester should preferably be set to about 1,000,000 at most.

The alginic ester should preferably be incorporated at an amount ranging from 5 to 15 parts by weight based on 100 parts by weight of the pulp. If the mixing amount of alginic ester is too small, the boding of pulp may become insufficient, thus making it difficult to retain the configuration of formed body. On the other hand, if the mixing amount of alginic ester is too large, it may be impossible to obtain a foam having a sufficient quantity of cells, resulting in a foam which is insufficient in terms of rigidity and flexibility. More preferably, the mixing amount of alginic ester should be confined to the range of 7 to 10 parts by weight based on 100 parts by weight of the pulp.

In addition to the above-mentioned components, the foaming composition according to this embodiment contains a surfactant. This surfactant acts to stabilize the cells formed by the expansion of the foaming composition. As the surfactant, it is possible to employ an ionic surfactant and a nonionic surfactant. As the ionic surfactant, it is possible to select from the group consisting of sodium stearate, sodium dodecyl sulfate, α-olefin sulfonate, sulfoalkyl amide, monocarboxy coco imidazoline compounds, dicarboxy coco imidazoline compounds, and sulfated aliphatic polyoxyethylene quaternary nitrogen compounds. On the other hand, the nonionic surfactant can be selected from the group consisting of octylphenol ethoxylate, modified linear aliphatic polyethers, and sorbitan esters. This surfactant can be optionally selected by taking into consideration water solubility, safety, biodegradability, etc.

The concentration of the surfactant should preferably be confined within the range of about 1% by weight to 10% by weight, more preferably, 3% by weight to 6% by weight based on a total weight of the foaming composition. If the concentration of the surfactant is less than 1% by weight, it may become difficult to obtain a sufficient effect of the surfactant. On the other hand, if the concentration the surfactant exceeds 10% by weight, the mechanical properties inherently conferred on foam material as well as the environmental adaptability of foam material may be degraded.

The foaming composition according to this embodiment may contain a plasticizing agent. This plasticizing agent acts to give flexibility to a foam material that has been bubbled and to minimize the shrinkage of the foam material during air blast drying process. As the plasticizing agent, it is possible to employ, for example, glycerol, glucose, polyhydric alcohol, triethanol amine, stearate, glycerin, diglycerin, triglycerin, pentaglycerin or decaglycerin.

The concentration of the plasticizing agent should preferably be confined to about 10% by weight or less based on a total weight of the foaming composition. If the concentration of the plasticizing agent exceeds 10% by weight, the mechanical properties inherently conferred on the foam material as well as the environmental adaptability of foam material may be degraded.

The foaming composition according to this embodiment may contain, as required, an oligomer foam-modifying agent or a polymer foam-modifying agent. When a foam modifying agent is incorporated in the foaming composition, the flexibility and toughness of foam material can be further enhanced. As the foam modifying agent, it is possible to employ polyethylene glycol, polyacryl amide, polyacrylic acid, polyvinyl alcohol, polyvinyl pyrrolidone, polyoxazoline or polyethylene imine.

As long as the concentration of the foam modifying agent is about 1% by weight based on a total weight of the foaming composition, it is possible to obtain the effects thereof. However, when the foam modifying agent is excessively incorporated in the foaming composition, the mechanical properties inherently conferred on the foam material as well as the environmental adaptability of foam material may be degraded. Therefore, the concentration of the foam modifying agent should preferably be confined to about 3% by weight at most based on the total weight of the foaming composition.

The foaming composition according to this embodiment may contain, as required, a foam-stabilizing agent. As the foam-stabilizing agent, it is possible to use, for example, ammonium stearate, dodecyl alcohol, tetradecanol, hexadecanol, tridecyloxy polyethanol, polyoxyethylated oleyl amine, etc. As long as the concentration of the foam-stabilizing agent is about 1% by weight based on a total weight of the foaming composition, it is possible to obtain the effects thereof. However, when the foam-stabilizing agent is excessively incorporated in the foaming composition, the mechanical properties inherently conferred on the foam material as well as the environmental adaptability of foam material may be degraded. Therefore, the concentration of the foam-stabilizing agent should preferably be confined to about 3% by weight at most based on a total weight of the foaming composition.

The foaming composition according to this embodiment may further contain, as required, an antioxidant, an ultraviolet ray absorbing agent, an antiseptic/mildew-proofing agent, a colorant, a perfume, an additive for plastics such as a flame retardant, etc. These additives should preferably be selected from those which are minimal in environmental load or impact.

A foaming composition according to this embodiment can be obtained by dispersing the aforementioned components including pulp, alginic ester, a surfactant, and, if required, a plasticizing agent in water in such a way as to obtain a suspension having a predetermined viscosity. A foam according to this embodiment can be obtained using this foaming composition.

In the manufacture of the foam, the foaming composition is mechanically stirred to bubble the foaming composition. This mechanical stirring may be effected using, for example, a pressure mixer, a continuous high-pressure foaming mixer, a kitchen mixer, a beater, or a homogenizer. The diameter of cells of the foam material thus obtained can be controlled by adjusting the duration of this bubbling. The foaming composition can be bubbled by blowing air into the foaming composition (so-called bubbling).

When the foaming composition is bubbled in this manner, a wet state of the foam material can be created. This wet state of the foam material may be referred to as a foam precursor. For example, this foam precursor can be flow-cast into a desired mold to mold it into a predetermined configuration. As for the thickness of the molded layer, it may be optionally selected depending on the application thereof, i.e., it may be as thin as about 1 mm or less or as thick as about 50 mm or more.

The foam according to this embodiment can be formed into a film or into a plate. Further, the foam may be formed into a shaped article by cast molding. By the term “shaped article”, it means works such as design and industrial arts and marchdizes that can be created by a man. These products can be manufactured by a press mold, an injection mold for plastic materials or a blow mold for plastic materials. The shaped article can be formed by the casting of a foaming composition using any of these molds.

After finishing the casting of a foaming composition according to this embodiment, the molded article is subjected to a drying treatment such as air blow drying or lyophilization to eliminate water from the molded article and reduce the water content thereof to 10% or less, thus manufacturing a foam having a desired fine cellular structure. This drying treatment can be performed by allowing the molded article to stand for a couple of days or so in an atmosphere of 0 to 50% in relative humidity and normal temperature (25° C.).

In view of saving the energy for drying without badly affecting the properties of foam, it is more preferable to perform the drying treatment of foam material by convection drying at normal temperature (25° C.). This convection drying at normal temperature can be realized using, for example, an apparatus (a bench ventilating device or a local exhaust system) which is capable of blowing air into a closed space.

Alternatively, the drying treatment can be performed by freezing a foamed composition at a temperature not higher than the melting point of water and then allowing the foamed composition to stand for all day long or so at a pressure close to vacuum. In this case, the water content of the foam material can be further reduced.

If the drying treatment is performed insufficiently, there may be raised a problem that a material which is vulnerable to moisture may be badly affected by the evaporation or discharge of water during the use thereof.

The foam according to this embodiment can be manufactured by drying a foam precursor under predetermined conditions. Namely, a binder is created by alginic ester and a surfactant, wherein cells each having a predetermined size due to the effects of the surfactant are enabled to disperse throughout the binder.

When the foam thus obtained is a film, it can be employed as it is, enabling it to be applied to a portion which is low in possibility of contacting with water or moisture such as an electronic apparatus, etc. The foam according to this embodiment may be used as a laminate comprising a plurality of foam sheets. More specifically, an adhesive such as a two-part epoxy adhesive, a rubber adhesive, cyanoacrylate adhesive, vinyl acetate resin emulsion, starch glue, etc. may be employed for fabricating the laminate. Alternatively, a film such as a resin film coated with a hot-melt adhesive, a polyimide adhesive film, ethylene/acrylate copolymer adhesive film, etc. may be interposed between foam sheets to fabricate a composite structure. These foam materials may be used as a single sheet or as a laminate and packed in a water resistant plastic bag, which is then applied to a portion which is low in possibility of contacting with water or moisture such as an electronic apparatus, etc.

The foam according to this embodiment can be used as a raw material for medical supplies, as an immobilized culture medium for cell culture, or as a raw material for packages to be employed in the industrial, agricultural and food fields (for example, food tray). Further, the foam according to this embodiment is expected to be effectively utilized, in the form of sheet or any optional configuration, as packaging vessels (one-tripper), toys, sheets, furniture components, building materials, car components, electric household appliances, components for office automation (OA) equipment, interior materials and housing materials.

The foam according to this embodiment may be degraded in function as a buffering material due to the compression thereof when it is used as a buffering material or as a structural material for a long period. If such a situation has happened to occur, the compressed foam can be regenerated by the method as explained below. Since the foam according to this embodiment includes alginic ester, it can be easily processed after the use thereof. The regeneration of the foam can be executed by a method comprising dissolving the foam in water to obtain a suspension; bubbling the suspension; and eliminating water from the suspension to make it into regenerated foam.

Specifically, water is added to foam to be regenerated and the resultant mixture is stirred by a stirrer equipped with agitating blades made, for example, of fluorinated resin (Teflon (registered trademark)) to dissolve the foam in water, thus obtaining an aqueous solution. On this occasion of dissolving foam in water, the quantity of water should desirably be adjusted so as to obtain an aqueous solution having a predetermined range of viscosity. As explained above, since the viscosity of this aqueous solution on the occasion of bubbling a suspension of foam has a great influence on the bubbled state and features of the foam to be obtained, it is necessary to control, in advance, the viscosity of aqueous solution. Irrespective of the ratio between the foam and water to be mixed with each other, the foam can be easily dissolved in water, taking several minutes to one hour. Namely, it does not take much time in dissolving the foam to be regenerated.

The dissolution of foam may be performed by a hot stirrer equipped with heater, thereby heating the aqueous mixture of foam up to about 60° C. By doing so, the dissolution of foam can be promoted. However, if the aqueous mixture of foam is excessively heated, the alginic ester contained in the foam may decrease in molecular weight thereof, thereby possibly causing the mechanical properties of foam to degrade as a whole. In order to prevent such a situation, the upper limit of heating temperature should desirably be limited to about 80° C.

Since the foam is transformed into a suspension in this method of regeneration, it is now possible to transport a large quantity of foam as compared with the conventional case where the foam is required to be transported as it is. Therefore, this regenerating method is advantageous on the occasion of regenerating and processing the foam. In the conventional method of transporting the foam, the quantity of foam to be transported is limited to about 15 wt % based on the maximum loading capacity of vehicle. Whereas, in the case of the foam according to this embodiment, not less than 15 wt % of foam can be transformed into a suspension. By suitably regulating the viscosity of suspension, the suspension can be bubbled to regenerate the foam. If it is impossible to regulate the viscosity of suspension to a predetermined range on the occasion of transporting the suspension, a virgin material may be mixed into the suspension immediately before bubbling the suspension. Alternatively, water may be added to the suspension so as to suitably regulate the viscosity of suspension, thereby making it possible to regenerate an excellent foam.

A foaming composition may be suspended in water to obtain a suspension having a predetermined viscosity and the resultant suspension may be treated according to the aforementioned process, thus manufacturing regenerated foam.

The foam thus regenerated is comparable in properties to unused foam (virgin material). Therefore, the regenerated foam can be employed in various fields in the same manner as the virgin material. For example, when the foam thus regenerated is a sheet, it can be employed as it is, enabling it to be applied to a portion which is low in possibility of contacting with water or moisture such as an electronic apparatus, etc. Alternatively, the foam sheet thus regenerated may be used as a laminate comprising a plurality of regenerated foam sheets. More specifically, an adhesive may be employed for fabricating the laminate. Alternatively, another kind of film may be interposed between regenerated foam sheets to fabricate a composite structure. These regenerated foam materials may be used as a single sheet or as a laminate and packed in a water resistant plastic bag, which is then applied to a portion which is low in possibility of contacting with water or moisture such as an electronic apparatus, etc.

As explained above, since the foam according to this embodiment is manufactured from a composition comprising pulp, alginic ester and a surfactant, the environmental load on the occasions of manufacturing the foam, recycling the foam, and scrapping of the foam can be minimized. Moreover, the foam according to this embodiment is excellent in buffering force and in restoring force. Furthermore, since the foam according to this embodiment is hydrophilic, it can be easily regenerated. The foam regenerated as described above is also excellent in buffering force and in restoring force just like the virgin material, thereby making it possible to recycle it as a buffering material.

Next, the present invention will be explained with reference to specific examples and comparative example as follows.

### EXAMPLE 1

As one example of alginic ester, there was prepared propylene glycol alginate (Kimiroid HV; Kimica Co., Ltd.; about 100,000 in weight average molecular weight (MW) and 0.92 g/cm³ in apparent density as a film). This alginic ester was dissolved in 74 g of water to prepare a 1 wt % aqueous solution of alginic ester.

10 g of pulp was prepared and added to the aqueous solution of propylene glycol alginate to obtain a mixed solution comprising 100 parts by weight of pulp and 9 parts by weight of propylene glycol alginate. Incidentally, the dry weight of the alginic ester in this mixed solution was 0.87 g. To 24.7 g of this mixed solution, there were added, as a surfactant, 0.6 g of sodium dodecyl sulfate (Wako Junyaku Industries Co.) and, as a plasticizing agent, 0.87 g of glycerin (Nakaraitesk Co., Ltd.), thus preparing the foaming composition of this example.

This foaming composition was stirred by a kitchen mixer to create a wet state of foam material. This foam material was then spread over a metal tray and allowed to dry, thereby manufacturing a foam. The drying of foam material was performed by applying an air flow to the foam material for two days at normal temperature. The foam thus dried was cut into pieces each having a size of 40 mm×40 mm to obtain a plurality of samples. These samples were then measured with respect to a total weight and an average thickness of three sheets thereof. As a result, the total weight was 1.8 g and the average thickness was 10.0 mm. The apparent density thereof, as calculated based on these values, was 0.03 to 0.05 g/cm³.

When water was added to the foam thus obtained so as make the concentration of alginic ester become about 1%, the foam was collapsed concurrent with the absorption of water by the foam, thus forming a suspension. Consequently, it was possible to confirm that the foam was sufficiently regenerative.

Further, the foam thus created was measured with respect to the compression strain thereof. More specifically, the foam was sliced and the sliced pieces were laminated to create a cubic body having a size of about 3 cm×3 m×3 cm, thus preparing a sample for measuring the compression strain. Then, this sample was compressed by applying a constant load (0.0625 kg/cm²) for a predetermined time (30 seconds) and then was released from the load. By measuring the height of the sample after 30 seconds later to calculate the ratio relative to the initial height of the sample, thus determining the strain. This measurement of strain was repeated five times, thus performing a repeated stress-strain test. The results of the repeated compression test are shown in the graph of FIGURE. In this graph, the ordinate represents a height recovery factor (100—strain) %. Among the values of abscissa, the even numbers represent the release of compression and the odd numbers represent the compression.

FIGURE shows that even though the height of sample was decreased as the sample was compressed, the height of sample was restored to the initial height as soon as the pressure to the sample was released.

The strain created after the five-times repeated compression test was less than 5%. The strain created after the five-times repeated compression test was employed as a criterion for the durable buffering property of sample, wherein the strain of less than 5% was marked by “◯” and the strain of 5% or more was marked by “Δ”.

Further, the mixing amount of components and the conditions of process were changed as follows in the preparation of the foaming compositions of Examples 2 to 7 and Comparative Example 1. Then, using these foaming compositions, various foams were created.

### EXAMPLES 2-4

Various foams were created by following the same process as described in Example 1 except that the mixing amount of components was changed as shown in the following Table 1.

### EXAMPLE 5

A foam was created by following the same process as described in Example 1 except that the surfactant was changed to sodium oleate.

### EXAMPLE 6

A foam was created by following the same process as described in Example 1 except that the drying method was changed to lyophilization.

### EXAMPLE 7

The foam obtained in Example 1 was dissolved in water for regenerating the foam, thus evaluating the regeneration property of the foam.

### COMPARATIVE EXAMPLE 1

A foaming composition was prepared by following the same process as described in Example 1 except that sodium alginic ester was substituted for propylene glycol alginate. The sodium alginic ester employed in this comparative example was Kimiroid HV (Kimica Co., Ltd.; about 100,000 in weight average molecular weight (MW), and 0.92 g/cm³ in apparent density as a film).

The durable buffering property of each of the foaming compositions of Examples 2-7 and Comparative Example 1 was evaluated in the same manner as described in Example 1, the results obtained are summarized in the following Table 1 together with the mixing amount of components and the density of foam.

TABLE 1

| | | Dry | Quantity | | | | | |
|---|---|---|---|---|---|---|---|---|
| | | weight of | of water | | Plasticizing | | | |
| | Pulp | alginate | used | Surfactant | agent | Density | | Buffering |
| | (g) | (g) | (g) | (g) | (g) | (g/cm³) | Drying method | durability |
| Example 1 | 10 | 0.87 | 74 | 0.6 | 0.87 | 0.03–0.05 | Dried at | ◯ |
| | | | | | | | normal temp. | |
| Example 2 | 10 | 0.87 | 74 | 0.6 | 0 | 0.04–0.05 | Dried at | ◯ |
| | | | | | | | normal temp. | |
| Example 3 | 10 | 0.87 | 54 | 0.6 | 0.87 | 0.07–0.08 | Dried at | ◯ |
| | | | | | | | normal temp. | |
| Example 4 | 10 | 0.87 | 74 | 0.6 | 0.87 | 0.04–0.05 | Dried at | ◯ |
| | | | | | | | normal temp. | |
| Example 5 | 10 | 0.87 | 74 | 0.6 | 0.87 | 0.03–0.05 | Dried at | ◯ |
| | | | | | | | normal temp. | |
| Example 6 | 10 | 0.87 | 74 | 0.6 | 0.87 | 0.03–0.04 | Freeze-drying | ◯ |
| Example 7 | 10 | 0.87 | 74 | 0.6 | 0.87 | 0.04–0.05 | Dried at | ◯ |
| | | | | | | | normal temp. | |
| Comp. | 10 | 1 | 74 | 0.6 | 0.87 | 0.03–0.05 | Dried at | Δ |
| Example 1 | | | | | | | normal temp. | |

The results of buffering property test conducted on the foam of Comparative Example 1 are shown in FIGURE. In the case of the foam of Comparative Example 1, once the foam was compressed, the restoring capability thereof was decreased. Therefore, it will be understood that the foam of Comparative Example 1 was poor in durability over a long period of use and hence unsuitable for practical use. Whereas, the foam of Example 1 indicated remarkable improvement in durability against the compression as compared with the foam of Comparative Example 1.

According to the present invention, it is possible to provide a foam which makes it possible to suppress the environmental load to a minimum on the occasions of manufacturing the foam, recycling the foam, and scrapping of the foam, and which is excellent in buffering force and restoring force and is suited for regeneration. According to the present invention, there are also provided a foaming composition for manufacturing such a foam and a method for manufacturing the foam.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A foaming composition comprising:
water;
pulp;
alginic ester; and
a surfactant.

2. The composition according to claim 1, wherein the pulp is selected from the group consisting of virgin pulp, regenerated pulp, bagasse pulp, and straw pulp.

3. The composition according to claim 1, wherein the pulp has a fiber length ranging from 1.5 to 5.5 mm, and a diameter ranging from 0.03 to 0.05 mm.

4. The composition according to claim 1, wherein the alginic ester is mixed therein at a mixing amount ranging from 5 parts by weight to 15 parts by weight based on 100 parts by weight of the pulp.

5. The composition according to claim 1, wherein the alginic ester is mixed therein at a mixing amount ranging from 7 parts by weight to 10 parts by weight based on 100 parts by weight of the pulp.

6. The composition according to claim 1, wherein the alginic ester is propylene glycol alginic ester.

7. The composition according to claim 6, wherein the propylene glycol alginic acid has a molecular weight ranging from 70,000 to 100,000.

8. The composition according to claim 1, wherein the surfactant is mixed therein at a concentration ranging from 1% by weight to 10% by weight based on a total weight of the composition.

9. The composition according to claim 1, wherein the surfactant is selected from the group consisting of sodium stearate, sodium dodecyl sulfate, α-olefin sulfonate, sulfoalkyl amide, monocarboxy-coco-imidazoline compounds, dicarboxy-coco-imidazoline compounds, sulfated aliphatic polyoxyethylene quaternary nitrogen compounds, octylphenol ethoxylate, modified linear aliphatic polyethers, and sorbitan esters.

10. The composition according to claim 1, further comprising a plasticizer.

11. The composition according to claim 10, wherein the plasticizer is selected from the group consisting of glycerol, glucose, polyhydric alcohol, triethanol amine, stearate, glycerin, diglycerin, triglycerin, pentaglycerin and decaglycerin.

12. A foam comprising:
pulp; and
a binder having cells, the binder binding the pulp and comprising alginic ester and a surfactant.

13. The foam according to claim 12, wherein the pulp is selected from the group consisting of virgin pulp, regenerated pulp, bagasse pulp, and straw pulp.

14. The foam according to claim 12, wherein the alginic ester is propylene glycol alginic acid.

15. The foam according to claim 12, wherein the surfactant is selected from the group consisting of sodium stearate, sodium dodecyl sulfate, α-olefin sulfonate, sulfoalkyl amide, monocarboxy-coco-imidazoline compounds, dicarboxy-coco-imidazoline compounds, sulfated aliphatic polyoxyethylene quaternary nitrogen compounds, octylphenol ethoxylate, modified linear aliphatic polyethers, and sorbitan esters.

16. The foam according to claim 12, wherein the binder further comprises a plasticizer.

17. A method for manufacturing a foam, comprising:
mixing pulp, alginic ester, a surfactant and water to obtain a foaming composition;
allowing the foaming composition to bubble, thereby obtaining a foam precursor; and
eliminating water from the foam precursor to form the foam.

18. The method according to claim 17, wherein the alginic ester is propylene glycol alginate.

19. The method according to claim 17, wherein eliminating water to form the foam comprises freezing the foaming composition to obtain a frozen foam; and drying the frozen foam.

20. The method according to claim 17, wherein eliminating water to form the foam comprises subjecting the foam precursor to convection-drying at normal temperature.
